(19) United States
(12) Patent Application Publication
(10) Pub. No.: US 2009/0227852 A1
(43) Pub. Date: Sep. 10, 2009

(54) PULSE SENSOR, PULSE METER, OXIMETER, JOYSTICK, AND HELMET

(76) Inventor: Eckard Glaser, Gerbrunn (DE)

Correspondence Address: KING & SCHICKLI, PLLC, 247 NORTH BROADWAY, LEXINGTON, KY, 40507, US

(21) Appl. No.: 12/093,654

(22) PCT Filed: Nov. 13, 2006
(86) PCT No.: PCT/DE2006/001987

§ 371 Date: Oct. 14, 2008

(30) Foreign Application Priority Data
Nov. 14, 2005 (DE) 10 2005 054 152.6

(51) Int. Cl. A61B 5/1455; A61B 5/02; A42B 1/08; H03K 17/94

(52) U.S. Cl. 600/324; 600/500; 2/424; 341/20

(57) ABSTRACT

This invention relates to a pulse sensor, comprising a radiation source (21, 22; 61, 62; 81, 82; 131, 132, 141, 142; 151, 152) for emitting photons and a photodetector (23; 63; 83; 133; 143; 153) for detecting the photons. The pulse sensor further comprises a holder (2, 3, 4; 53, 54; 130; 150) for fixing the radiation source and the photodetector to a person. Moreover, the pulse sensor comprises a connection (55; 165) to a pulse meter, wherein the pulse meter controls the radiation source (21, 22; 61, 62; 81, 82; 131, 132, 141, 142; 151, 152) and reads out the photodetector (23; 63; 83; 133; 143; 153) via the connection (55; 165). The pulse sensor finally comprises an stimulus generator (15, 16, 24, 41, 43, 44, 45; 52, 64; 75, 76, 84, 85; 134, 135, 144, 145; 156) attracting the attention of the person when the stimulus generator is activated by the pulse meter because the pulse of the person is outside a permitted range (107). Also, this invention relates to oximeters, joysticks and helmets.

The invention relates to the technical field of pulse sensors, pulse meters and oximeters as well as to applications of pulse sensors in joysticks and helmets. Specifically, the invention relates to pulse and/or oximeters of small structural shapes and to sensors for such apparatus, which are equipped with an acoustic and/or optical alarm.

The basic mode of operation of oximeters is described, for example, in U.S. Pat. No. 6,736,759 B1. Oxyhemoglobin found in oxygen-enriched corpuscles and deoxyhemoglobin found in low-oxygen corpuscles have a different optical absorption. Deoxyhemoglobin absorbs more in the red range, while oxyhemoglobin absorbs more in the infrared range. Absorption curves are shown, for example, in U.S. Pat. No. 5,152,296. To detect these differences, mostly red light-emitting diodes, which emit light mainly between 610 nm and 650 nm, and infrared light-emitting diodes having an emission mainly between 810 and 850 nm, are used.

Light having two different wavelengths is alternately directed at tissue supplied with blood. A photosensor detects transmitted or backscattered light, depending on the model of the oximeter sensor. Depending on which of the light-emitting diodes lights up, the absorption or backscattering in the red or infrared range is measured.

The absorption of light in the tissue also depends on the phase of the heartbeat. During the systole the amount of blood in the arteries increases, which leads to a detectable increased light absorption. Therefore, the pulse of a subject can be determined from the time interval between the light absorption peaks. Although this type of pulse measurement only requires one wavelength, the pulse can also be determined from the signal of an oximeter sensor. Therefore, the additional effort for a pulse measurement in an oximeter is small. Consequently, almost exclusively pulse oximeters are being sold at present.

U.S. Pat. No. 6,736,759 B1 describes several embodiments of a training monitoring system. This system comprises an electronic positioning apparatus operated on a GPS basis (global positioning system), a physiological monitor, which may include an oximeter, as well as a display device. In one embodiment, a GPS module, an oximeter, batteries, a processor-transmitter module and other modules may be attached to a belt worn by a sportsman around his belly. On one wrist the sportsman wears the display device, which is received in a watch-like housing. The display device comprises a (radio) receiver so as to receive data from the processor-transmitter module, a processor, a display screen, input switches as well as one or more alarms delivering an audible and/or visible information such as blinking. An alarm can, for example, be activated when the blood oxygen content or the pulse are outside a predetermined range. The predetermined range can be adjusted by means of the input switches. In another embodiment, the display device can be accommodated in a tacho-like housing, which is attached to a handlebar of a bicycle. Finally, an embodiment is described, in which the data to be displayed are played into the eyes of a user via spectacle glasses.

In U.S. Pat. No. 5,490,523 a finger clip pulse oximeter is described, which is sold by Nonin Medical Inc. obviously under the product name 9500 Onyx. In use, for example, the index is clamped between a lower housing and an upper housing. A spring presses both housings against the finger. In the lower housing there is provided a battery case and an aperture through which two light-emitting diodes (LEDs) can emit light. In the upper housing there is substantially accommodated a board carrying the electronic system and six 7-segment LEDs for displaying the measured values. The upper half of the upper housing is transparent. On the underside of the board a photodetector is mounted, which picks up through an aperture in the lower half of the upper housing the light transmitted by the finger. A flat ribbon cable connects the battery case, the two LEDs mounted on the flat ribbon cable, the upper half of the lower housing, the lower half of the upper housing and the board. In a second embodiment a reflection pulse oximeter may be used, which is described, for example, in U.S. Pat. No. 5,224,478. In this embodiment, all units may be accommodated in one housing. For the measurement, the user places his finger into a hollow provided in said housing. In a third embodiment, the shape of the housing of a reflection pulse oximeter can be adapted to the shape of the forehead of a user and is fixed to the forehead presumably by means of a tape.

Under the product name 3100 WristOx Nonin Medical Inc. offers a pulse oximeter in watch form, which may be worn on one wrist. The 3100 WristOx can be connected, for example, to a finger clip sensor.

A similar device is described in U.S. Pat. No. 6,731,962 B1 and is called a finger oximeter. It is sold, for example, by Miami Medical under the trademark DIGIT and is obviously manufactured by BCI. In addition to the finger clip pulse oximeter described in U.S. Pat. No. 5,490,523 the finger oximeter includes a bi-directional radio interface, so that the finger oximeter can communicate in a cordless manner with a remote monitoring system, e.g. a Vital Signs of Smith Medical PM, Inc., Waukesha, Wis., USA. The signal received can be displayed graphically or alphanumerically in the monitor. The signal can also be outputted by the monitoring system as an audio or visual alarm if an undesired threshold is reached or exceeded. The radio interface can use the Bluetooth protocol. The finger oximeter can be activated or deactivated by a switch provided in the housing of the finger oximeter or by the monitoring system.

In U.S. Pat. No. 5,800,349 different transmission pulse oximeter sensors are described, in which the light sources and the photodetectors are not arranged on one axis; but offset with respect to each other.

In U.S. Pat. No. 6,771,172 B1 a portable patient monitor is described, in the handle of which red and yellow light-emitting diodes are installed so as to output visual alarms. An alarm may also be outputted by a loudspeaker. The alarm thresholds can be set by means of a keyboard. The patient monitor can be a portable oximeter.

If the oxygen partial pressure in the respiratory air decreases, humans lose their consciousness without realizing it. The time until the loss of consciousness is called “usable consciousness”. The oxygen partial pressure can drop, for example, because of a defect in the air conditioning system of aircraft or if gliders flying too high. Whether and when a specific oxygen partial pressure leads to a loss of consciousness also depends on the constitution of the person concerned. An imminent loss of consciousness can be detected by means of an oximeter from the oxyhemoglobin content of the person more independently of its constitution than it would be possible, for example, by measuring the pressure or the oxygen partial pressure in the ambient air. After all, persons with pulmonary diseases can have problems already in 1500 to 2000 m above mean sea level, e.g. during mountain tours.

If during the usable consciousness the risk of a loss of consciousness is displayed to the persons, they can take countermeasures.

It is the object of the invention to provide pulse and oximeter sensors, pulse and oximeters as well as joysticks and helmets for active persons.

This object is achieved with the teaching defined in the independent claims.

Preferred embodiments of the invention are defined in the dependent claims.

It is an advantage of the invention that the attention of sportsmen, pilots or persons having pulmonary diseases is actively drawn to an overload or oxygen deficit in their body.

A buzzer, a loudspeaker, a bright light source, a vibrator or electrodes have the advantage that all of these means actively attract the attention of a person. Thus, the person is not forced to check one or two three-digit 7-segment displays from time to time. Besides, the invention solves so-to-speak also the problem that the time interval between two checks must be shorter than the usable consciousness. When using apparatus according to the prior art, the person would, after all, have to be reminded somehow of the next reading of the display.

A battery-operated pulse meter can be equipped with a vibration alarm in a particularly compact manner if one or more batteries are moved with respect to the housing.

It is an advantage of the use of a warning and alarm mode that the urgency of a danger is recognizable.

It is particularly advantageous to draw a person's attention to too low an oxyhemoglobin content in its blood by means of an stimulus, because there is the danger that the person loses its consciousness without pains or malaise.

Advantageously, a pulse sensor or an oximeter sensor can be integrated in a joystick or a helmet.

Also stimulus generators such as LEDs or electrodes can advantageously be integrated in joysticks or helmets.

A preferred embodiment of the invention will be explained in more detail below by means of the accompanying drawings, wherein

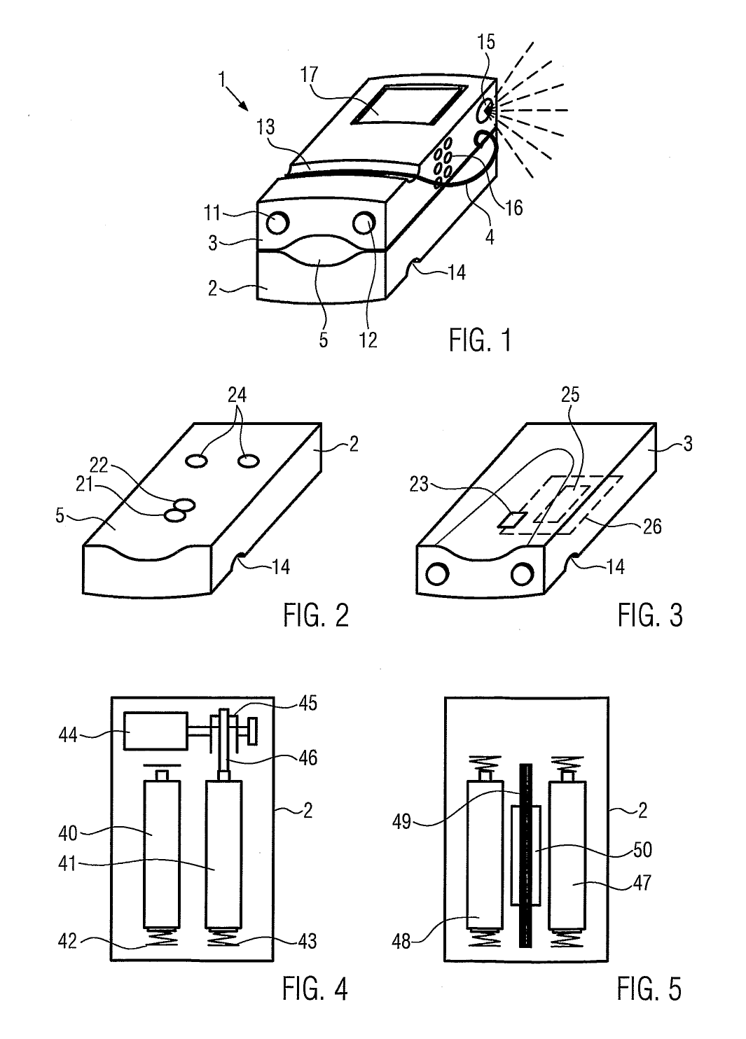

FIG. 1 shows a finger clip pulse oximeter according to the invention;

FIG. 2 shows a lower half of the housing of the finger clip pulse oximeter illustrated in FIG. 1;

FIG. 3 shows an upper half of the housing of the finger clip pulse oximeter illustrated in FIG. 1;

FIG. 4 shows a section through a first embodiment of the lower half of the housing illustrated in FIG. 2;

FIG. 5 shows a section through a second embodiment of the lower half of the housing illustrated in FIG. 2;

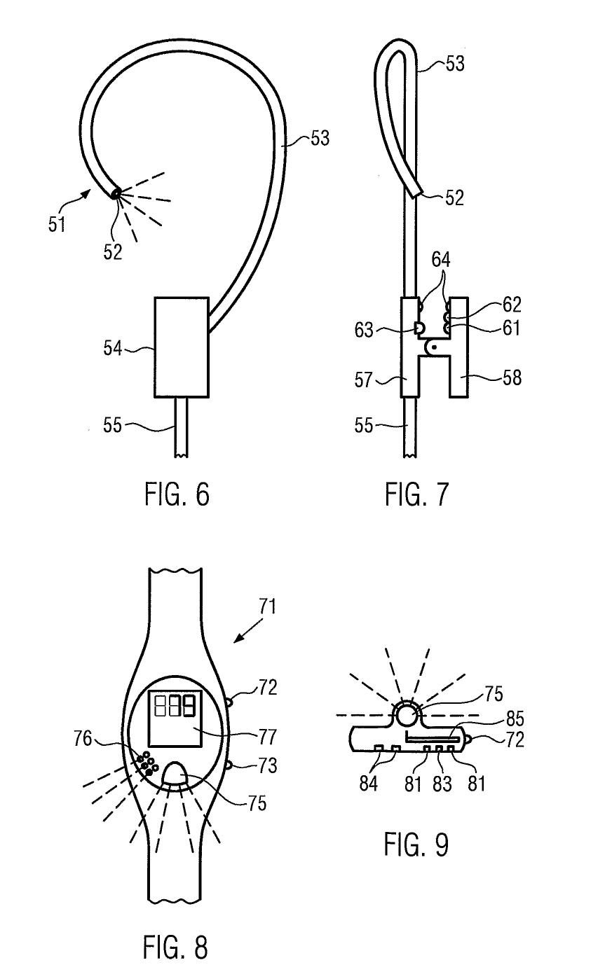

FIG. 6 shows a lateral view of an ear clip sensor according to the invention;

FIG. 7 shows a rear view of an ear clip sensor according to the invention;

FIG. 8 shows a top view of a wrist pulse oximeter according to the invention;

FIG. 9 shows a section through the wrist pulse oximeter illustrated in FIG. 8;

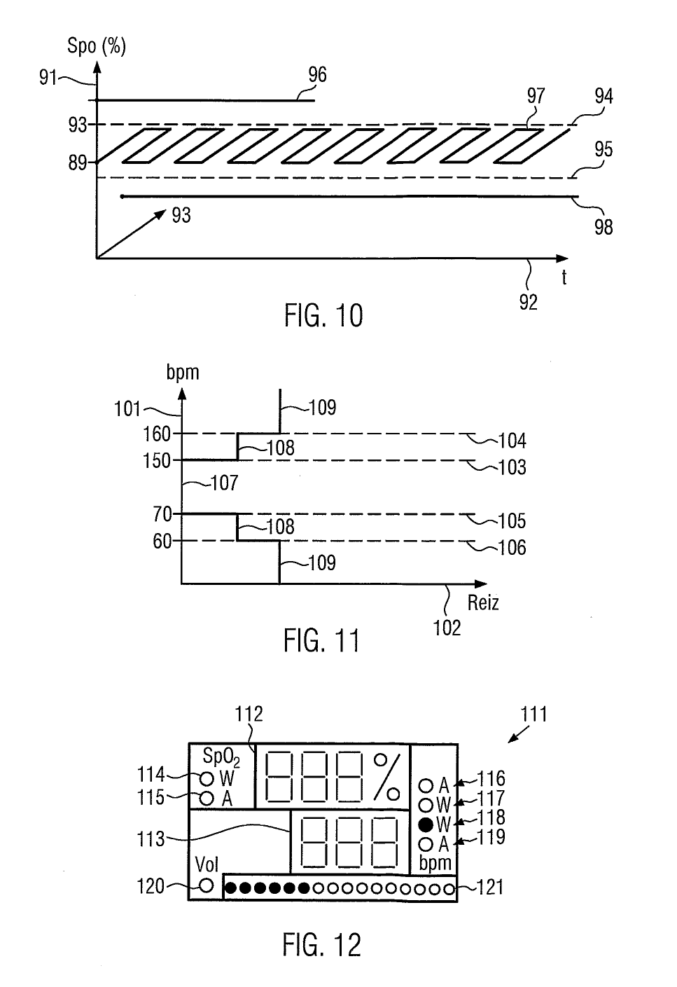

FIG. 10 shows a warning and alarm characteristic of an oximeter according to the invention;

FIG. 11 shows a warning and alarm characteristic of a pulse meter according to the invention;

FIG. 12 shows a display according to the invention;

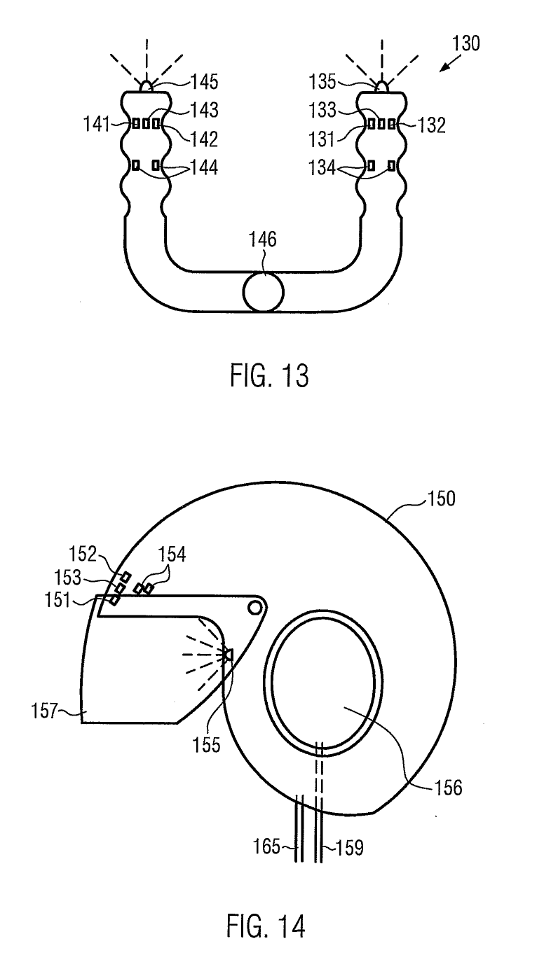

FIG. 13 shows a joystick according to the invention; and

FIG. 14 shows a helmet according to the invention.

One essential aspect of this invention consists in the measurement of the oxyhemoglobin content in the blood of a person and/or the pulse of the person and in warning the person by appropriate stimuli if the oxyhemoglobin content or the pulse adopt undesired or even dangerous values.

The finger clip pulse oximeter 1 shown in FIG. 1 comprises an upper housing half 3, a lower housing half 2 as well as a spring 4 pressing the upper housing half 3 and the lower housing half 3 together. The spring 4 engages into the grooves 13 and 14 in the upper housing half 3 and the lower housing half 2, respectively. Both the upper housing half 3 and the lower housing half 2 include a recess 5 for receiving a finger, especially an index, whereby the depth of the recess 5 is dimensioned in such a close-fitting manner that the upper and the lower housing halves 3 and 2 are not pressed against each other, but press against the finger.

The upper housing half 3 comprises a first key 11 and a second key 12 for controlling the pulse oximeter 1, as well as a groove 13 into which the spring 4 is engaged. The operation by means of the two keys 11 and 12 shall be explained in more detail in connection with FIGS. 10 to 12. The upper housing half 3 moreover comprises a display 17 as well as several stimulus generators.

The stimulus generators include a bright LED 15, a buzzer 16 behind buzzer apertures, electrodes 24 and vibrators. The latter shall be explained in more detail in connection with FIGS. 4 and 5. These stimulus generators have the property that they are able to actively draw the user's attention to them. 7-segment displays, as are employed, for example, in the 9500 Onyx and in the DIGIT, do not represent stimulus generators in accordance with this application. One could also say that stimulus generators in accordance with this application are non-alphanumeric displays. However, this does not so clearly express the purpose of actively attracting the user's attention, and it does not include all of the embodiments according to the invention either, as will be explained below three paragraphs from here.

Usually, such 7-segment displays are multiplexed with a frequency of such an intensity that the human eye perceives a uniform light. Thus, the brightness fluctuates between 2/7 for a “1” and 7/7 for an “8”. In the case of two-digit displays and in the interesting range between 70% and 100% of the oxyhemoglobin content the fluctuations in the brightness on representing the numbers between 70 and 99 are far smaller. In addition, the numbers of the low-order position are periodically passed through three times, which is not suited to display the falling below a threshold.

Finally, the 7-segment displays in the 9500 Onyx and the DIGIT are oriented such that the displays are not visible in a relaxed position of the hand or during a number of activities such as driving, flying or hiking.

In order to transform conventional 7-segment displays into stimulus generators in accordance with this application, for example, the 7-segment display would have to be switched on and off in an alarm or warning range in a manner visible to the human eye, that is at a frequency between approximately 0.5 and 20 Hz, (compare FIGS. 9 and 10). Alternatively or additionally, the 7-segment display could be switched off entirely in the green range 107. Furthermore, the 7-segment display should be accommodated on the side of the finger clip pulse oximeter 1 next to the buzzer 16.

FIG. 2 shows the lower housing half 2 and, specifically, the upper side thereof with the recess 5. On the upper side one can see a light-emitting diode (LED) 21, an infrared light-emitting diode (IRED) 22 and two electrodes 24. The LED 21 together with IRED 22 form a radiation source emitting at least two light wavelengths. As will be explained in connection with FIG. 4 below, the lower housing half 2 includes batteries or accumulators serving as energy sources. The electrodes 24 serve to generate by means of current pulses another type of stimulus alternatively or in addition to light and sound, so as to actively warn or alert the user.

FIG. 3 shows the upper housing half 3 and specifically the lower side thereof with the recess 5. A photodetector 23 can be seen on the lower side. The photodetector 23 together with the LED 21 and the IRED 22 form an oximeter sensor in the narrower sense. The upper housing half 3 specifically comprises a board 26 with the evaluation circuit 25. The lower and upper housing halves 2 and 3 are connected, for example, by a (non-illustrated) flat ribbon cable as to supply the evaluation circuit 25 with energy and control the LED 21, the IRED 22 and the electrodes 24.

In operation, the photodetector converts the red and infrared photons transmitted by the finger into electric signals as to determine in a manner known per se the content of oxyhemoglobin. The photodetector can be mounted opposite the LED 21 and the IRED 22 (compare U.S. Pat. No. 5,490,523 and U.S. Pat. No. 6,731,962) or offset opposite to the LED 21 and the IRED 22 (compare U.S. Pat. No. 5,800,349). To distinguish between red and infrared, the LED 21 and IRED 22 can be switched on in a phase-shifted manner. In another embodiment, the LED 21, the IRED 22 and the photodetector 23 may be accommodated in the same housing half, so that the oxyhemoglobin content is determined by means of backscattered photons (U.S. Pat. No. 5,490,523 “reflective type pulse oximeter”). In this connection, one also talks about a reflection pulse oximeter or reflection sensor.

FIG. 4 shows a section through an embodiment of the lower housing half 2. As was mentioned above, the lower housing half 2 includes above all the batteries 40 and 41 serving as energy sources. A spring 42 presses in a usual manner the battery 40 against a contact on the opposite side. The battery 41 moreover serves as inertial mass as to make the lower housing half 2 and thus also the upper housing half 3 vibrate. To this end, a motor 44 drives an eccentric 45, on which a contact 46 is rotatably mounted. The contact moves the battery 41 with respect to the lower housing half 2 when the motor 44 with the eccentric 45 is rotated. The spring 43 thereby presses the battery 41 against the contact 46, wherein the spring 43 is pressed together alternately stronger or less strongly.

FIG. 5 shows a section through another embodiment of the lower housing half 2. Again, the lower housing half 2 comprises two batteries 47 and 48, which are held in the lower housing half by means of springs on both ends. A coil 49 is accommodated between both batteries. Inside the coil there is located a mobile magnet 50, which may be fixed to the housing by a bendable tongue. It is irrelevant as to whether the magnet 50 is clamped between the two batteries 47 and 48 free from play, or whether—as is shown in FIG. 5—a small gap is provided between the batteries 47 and 48 and the magnet 50.

Depending on the direction of the current flow in the coil 49, the magnet 50 is pressed against the right battery 47 or the left battery 48 and moves the same. The batteries 47 and 48 experience a particularly great mechanical deflection if the coil 49 is operated with an alternating current, whose frequency corresponds to the mechanical resonance frequency of the batteries 47 and 48. The mechanical resonance frequency of the batteries should, again, correspond to the desired vibration frequency.

In another embodiment, the batteries may be fixed by a spring to be mobile on one side only, or in the conventional manner, that is immobile with respect to the lower housing half 2. In the latter case, then merely the magnet 50 serves as inertial mass.

The magnet 50 may be both a permanent magnet and an electromagnet, e.g. including a soft iron core.

FIG. 6 shows a lateral view of an inventive ear clip sensor 51. FIG. 6 shows a rear view of the ear clip sensor 51. The ear clip sensor 51 comprises a clamp 54 for clamping an earlobe of a user. To increase the wearing comfort and to ensure a stable seat of the ear clip sensor 51 the ear clip sensor includes an ear bow 53 which, similar to a hearing aid, extends between the outer ear and the head upwardly and finally forwardly so as to transfer a force onto the outer ear. The ear bow 53 may be made of a thermoplastic material so as to adopt the shape of the ear bow to the anatomy of the user. The ear bow 53 may be hollow and comprise a sound aperture 52 on its end to warn or alert a user. The buzzer or loudspeaker for the generation of sound can be accommodated for example, in the inner half of the clamp 57 or in a thickened portion of the ear bow 53.

In the outer half of the clamp 68 there are mounted an LED 61 and an IRED 62. A photodetector 63 is accommodated in the inner half of the clamp 57. The photodetector 63 can be located either opposite the LED 61 and the IRED 62, or can be mounted to be offset opposite to the LED 61 and the IRED 62. In other embodiments, the LED 61, the IRED 62 and the photodetector 63 can be accommodated together either in the inner clamp 57 or the outer clamp 58 so as to determine the oxyhemoglobin content by means of the backscattering (reflection type).

Electrodes 64 mounted in the inner half of the clamp 57 and/or the outer half of the clamp 58 to touch, during the use, the skin of a user, specifically his earlobe, serve to warn or alert the user by means of current pulses.

An electric line 55 serves to connect the ear clip sensor 51 to a pulse oximeter. To achieve a better stability and higher wearing comfort the line 55 is fixed to the inner half of the clamp 57. Via the line 55 the LED 61, the IRED 62 and the electrodes 63 are controlled and the photodetector 63 is read out.

In another embodiment the halves of the clamp 57 and 58 may also accommodate batteries, an evaluation circuit, possibly even keys and a display for adjusting warning and alarm thresholds, thereby creating an ear clip pulse oximeter. In this case, the line 55 is dispensable.

FIG. 8 shows a top view of an inventive wrist pulse oximeter 71. FIG. 9 shows a section through the wrist pulse oximeter 71. The wrist pulse oximeter 71 has the shape of a watch and is correspondingly worn on the wrist. It comprises the same stimulus generating means as the finger clip pulse oximeter 1: a bright LED 75, a buzzer 76, an eccentric 85, which simultaneously serves as inertial mass so as to make the wrist pulse oximeter 71 vibrate, as well as electrodes 84 to generate current pulses. As is shown in FIG. 9, the lower side of the wrist pulse oximeter 71, which faces the wrist of the user, is provided with the reflection oximeter sensor including the LED 81, the IRED 82 and the photosensor 83.

On the upper side of the wrist pulse oximeter 71 there is moreover located a display 77, which can be designed, for example, like the display 111 illustrated in FIG. 11. On a flat side of the wrist pulse oximeter 71 there are mounted a first key 72 and a second key 73, by means of which, for example, warning and alarm limit values can be inputted.

In another embodiment, the wrist pulse oximeter 71 can also be provided with a finger clip sensor similar to the 3100 WristOx, which is connected to the wrist pulse oximeter by an electric cable. In this case, the wrist pulse oximeter can serve above all as a mechanical retaining device for the finger clip sensor so as not to get lost during work or practicing sports. The finger clip sensor may be smaller and lighter than a finger clip pulse oximeter because the batteries and the evaluation circuit can be accommodated in another housing. This increases the wearing comfort and reduces the risk of losing the finger clip sensor. In this embodiment, the stimulus generator or generators can be located in the wrist pulse oximeter and/or the finger clip sensor.

FIG. 10 shows the warning and alarm system for an inventive oximeter. In this three-dimensional diagram the oxyhemoglobin content is plotted on the SpO-axis 91, the time (t) on the time axis 92 and the stimulus intensity generated by an stimulus generator on the stimulus axis 93. In the diagram shown in FIG. 10, a warning threshold 94 is plotted at 93% of the oxyhemoglobin content and an alarm threshold 95 at 89% of the oxyhemoglobin content.

If the oxyhemoglobin content is above the warning threshold 94, no stimulus is generated, which is illustrated by line 96. If the oxyhemoglobin content is below the warning threshold 94, but above the alarm threshold 95, a warning stimulus 97 is outputted. This may be formed by periodically switching an stimulus on and off. The frequency or the pulse duty factor of the on-off switching, the frequency of a buzzer tone or a vibrator, the volume of the buzzer tone or the intensity of the vibrations may be chosen on the basis of the oxyhemoglobin content, thereby allowing to vary the intensity of a warning. If the oxyhemoglobin content falls below the alarm threshold 95, an alarm by the stimulus generator or generators is outputted. The alarm may consist of a continuous stimulus, which is shown by line 98.

It is known that especially at the edge of the vision range the human eye is more sensitive to changes in brightness and color, which are normally caused by movements in the environment. Therefore a blinking LED is suited better to attract the attention of a user than an LED which is switched on. In another embodiment especially with respect to optical stimuli, therefore, the warning can be represented by a switched on LED and the alarm by a blinking LED.

FIG. 11 shows the warning and alarm system for a pulse meter. On the pulse axis 101 the pulse is illustrated in beats per minute (bpm). On the stimulus axis 102 the stimulus intensity is represented. Moreover represented are a lower alarm threshold 106 at 60 bpm, a lower warning threshold 105 at 70 bpm, an upper warning threshold 103 at 150 bpm and an upper alarm threshold 104 at 160 bpm. Between the lower warning threshold 105 and the upper warning threshold 103 the green range 107 is provided, in which no stimulus is generated. If the pulse ranges between the lower alarm threshold 106 and the lower warning threshold 105 or between the upper warning threshold 103 and the upper alarm threshold 104 a warning 108 is outputted. If the pulse falls below the lower alarm threshold 106 or exceeds the upper alarm threshold 104, an alarm 109 having about double the stimulus intensity of a warning 108 is outputted.

Similar to oximeters, the frequency or the pulse duty factor of the on-off switching of an stimulus, the frequency of a buzzer tone or a vibrator, the volume of the buzzer tone or the intensity of the vibrations may be chosen on the basis of the interval of the measured pulse from the green range 107.

In one embodiment, the pulse meter, the oximeter or pulse oximeter can be manufactured with predefined warning and alarm thresholds. If the warning and alarm thresholds are to be adjustable, a display of the adjusted warning and alarm threshold is helpful. This can be readily achieved by a potentiometer with a scale to make the potentiometer position readable. Expediently, the scale shows the adjusted warning and alarm thresholds immediately.

However, the embodiments shown in FIGS. 1 to 9 make use of two keys for adjusting the warning and alarm thresholds as well as a display for controlling the warning and alarm thresholds.

The pulse oximeters may have different modes of operation. In one mode parameters such as the warning and alarm thresholds may be adjusted, in the other one the measured values are displayed. The displays 17 and 77 shown in FIGS. 1 and 8 may be designed like the display 111 shown in FIG. 12.

The display 111 comprises an oxyhemoglobin section 112, a pulse section 113, various indicators 114 to 120 as well as a volume bar 121. In the oxyhemoglobin section 112 either the oxyhemoglobin concentration or the warning or alarm threshold 94 or 95, respectively, are displayed. In the pulse section 113 either the pulse or the lower or upper warning and alarm threshold 104, 103, 105, 106 are displayed in bpm. The volume bar 121 generally shows the intensity of an stimulus in the event of an alarm or warning. Specifically, the volume of a buzzer 16 or 76 is envisaged. It is important that the stimulus intensity, that is the volume, cannot be set to zero because this would be equal to deactivating a warning.

As to what is displayed where, and what meaning the two keys have, is shown by the indicators 114 through 120. If none of the indicators is dark, like indicator 118 in FIG. 12, the apparatus in the measurement mode and the oxyhemoglobin concentration is displayed in the oxyhemoglobin section 112, the pulse is displayed in the pulse section 113 and the volume is displayed in the volume bar 121. By briefly pressing the first key 11 or 72 the apparatus is transferred into the setting mode. Now, first the indicator 114 turns dark. Each further brief pressing of the first key results in a cyclic advancement from indicator to indicator. If the first key is pressed long, altered warning and alarm thresholds are stored and used in the later operation. If no key is pressed for 10 seconds, the apparatus returns to the measurement mode without storing new warning or alarm thresholds. On pressing both keys, the apparatus is switched on or off.

On pressing the second key the parameter just selected, which is displayed by the corresponding indicator, is cyclically advanced by one step. If the second key remains pressed down, the parameter selected can be advanced after 3 seconds by one step per second. The advancing frequency can be increased with the duration of maintaining the second key in a pressed position.

The warning or alarm value corresponding to the dark indicator is displayed in the corresponding section and can be altered by pressing the second key. The “A” right of an indicator stands for alarm threshold. The “W” right of an indicator stands for warning threshold. The arrangement of the indicators in the display 111 corresponds to the arrangement of the warning and alarm thresholds in FIGS. 10 and 11.

If one of the indicators 114 or 115 is dark, the level of the warning threshold 94 or the level of the alarm threshold 95, respectively, is displayed in the oxyhemoglobin section 112 and can be altered by pressing the second key. The warning threshold can thereby be set in 1%-steps between 100% of the oxyhemoglobin content and the alarm threshold. The alarm threshold can be set in 1%-steps between 100% and 70% of the hemoglobin content.

If one of the indicators 116, 117, 118 or 119 is dark, the level of the upper alarm threshold 104, of the upper warning threshold 103, of the lower warning threshold 105 and the lower alarm threshold 106, respectively, can be adjusted. The lower alarm threshold can be adjusted in a range of 40 bpm to 230 bpm. The upper alarm threshold can be adjusted in a range from the lower alarm threshold to 230 bpm. The lower warning threshold can be adjusted in a range from the lower alarm threshold to the upper alarm threshold. The upper warning threshold can be adjusted in a range from the lower warning threshold to the upper alarm threshold.

If the indicator 120 is dark, the volume can be adjusted by pressing the second key. The control range can range from 10% to 100% of the maximally obtainable volume bars. In addition, there may be provided other bars and corresponding indicators for the adjustment of the other stimuli, that is the brightness of the LED, the intensity of the current pulses and the intensity or frequency of the vibrations, with the aid of the second key. With respect to the stimuli, especially the volume, it is recommendable to select the step sizes logarithmically.

FIG. 13 shows a joystick 130 according to the invention for aircraft and helicopters. In each horn of the joystick 130 there are accommodated a reflection pulse oximeter sensor comprising the LEDs 131 and 141, respectively, the IREDs 132 and 142, respectively, and the photodetectors 133 and 143, respectively. Besides, each horn includes electrodes 134 and 144 for generating electric stimuli. Moreover, a bright LED 135 and 145, respectively, for generating optical stimuli is provided on each horn. The actual pulse oximeter can be mounted outside the joystick 130. Connecting cables may be passed through the joystick bearing 146. The pulse oximeter may be connected to the sound system of the aircraft or helicopter, which substantially consists of a transceiver and perhaps an intercom system. Thus, warnings or alarms can also be issued via the headset worn by the pilots.

Similar to the joystick 130 pulse oximeters can also be mounted on their own or in combination with stimulus generators in other operating elements, such as bicycle or motorcycle handlebars or steering wheels for cars and commercial vehicles.

FIG. 14 shows a helmet 150 according to the invention. The helmet serves as a holder for a reflection pulse oximeter sensor, which consists of the Led 151, the IRED 152 and the photodetector 153 and measures the oxyhemoglobin content in the forehead of the pilot. Electrodes 154 can generate electric stimuli. The LED 155 together with the visor 157 can generate optical stimuli. The reflection pulse oximeter sensor, the electrodes 154 and the LED 155 are connected by an electric line 165 to an appropriate pulse oximeter, which may be connected to the sound system of the aircraft or helicopter so as to issue acoustic warnings or alarms via the line 159 and the headset earpiece 156.

Similar to the helmet 150 pulse oximeters can also be mounted on their own or in combination with stimulus generators in other articles of clothing, e.g. gloves or boots.

Above, the invention was explained in more detail by means of preferred embodiments. A person skilled in the art will appreciate, however, that various alterations and modifications may be made without departing from the spirit of the invention. Therefore, the scope of protection will be defined by the claims and their equivalents set forth below.

### LIST OF REFERENCE NUMBERS

1 finger clip pulse oximeter
2 lower housing half
3 upper housing half
4 spring
5 recess
11, 12 keys
13, 14 groove
15 LED
16 buzzer
17 display
21 LED
22 IRED
23 photodetector
24 electrodes
25 evaluation circuit
26 board
40, 41 battery
42, 43 spring
44 motor
45 eccentric
46 contact
47, 48 battery
49 coil
50 magnet
51 ear clip sensor
52 sound aperture
53 ear bow
54 clamp
55 line
57 inner half of clamp
58 outer half of clamp
61 LED
62 IRED
63 photodetector
64 electrodes
71 wrist pulse oximeter
72, 73 key
75 LED
76 buzzer apertures
77 display
81 LED
82 IRED
83 photosensor
84 electrodes
85 eccentric
91 SpO-axis
92 time axis
93 stimulus axis
94 warning threshold
95 alarm threshold
96 no stimulus
97 warning stimulus
98 alarm
101 pulse axis
102 stimulus axis
103 upper warning threshold
104 upper alarm threshold
105 lower warning threshold
106 lower alarm threshold
107 green range
108 warning
109 alarm
111 display
112 oxyhemoglobin section
113 pulse section
114-120 indicators
121 volume bar
130 joystick
131, 141 LED
132, 142 IRED
133, 143 photodetector
134, 144 electrodes
135, 145 LED
146 joystick bearing
150 helmet
151 LED
152 IRED
153 photodetector
154 electrodes
155 LED
156 headset earpiece
157 visor
159, 165 line

1. A pulse sensor, comprising:
a radiation source (21, 22; 61, 62; 81, 82; 131, 132, 141, 142; 151, 152) for emitting photons;
a photodetector (23; 63; 83; 133; 143; 153) for detecting the photons;
a holder (2, 3, 4; 53, 54; 130; 150) for fixing the radiation source and the photodetector to a person; and
a connection (55; 165) to a pulse meter, wherein the pulse meter controls the radiation source (21, 22; 61, 62; 81, 82; 131, 132, 141, 142; 151, 152) and reads out the photodetector (23; 63; 83; 133; 143; 153) via the connection (55; 165),
characterized by
an stimulus generator (15, 16, 24, 41, 43, 44, 45; 52, 64; 75, 76, 84, 85; 134, 135, 144, 145; 154, 155, 156) for generating an stimulus attracting the attention of the person when the stimulus generator is activated by the pulse meter, wherein the stimulus generator (15, 16, 24, 41, 43, 44, 45; 52, 64; 75, 76, 84, 85; 134, 135, 144, 145; 154, 155, 156) is activated by the pulse meter when the pulse of the person is outside a permitted range (107).

2. The pulse sensor according to claim 1, characterized in that the stimulus generator is a buzzer (16, 52, 76).

3. The pulse sensor according to one of the preceding claims, characterized in that the stimulus generator is a loudspeaker (156).

4. The pulse sensor according to one of the preceding claims, characterized in that the stimulus generator is a bright LED (15; 75; 135, 145; 155).

5. The pulse sensor according to one of the preceding claims, characterized in that the stimulus generator is a vibrator (43, 44, 45; 85).

6. The pulse sensor according to one of the preceding claims, characterized in that the stimulus generator comprises two electrodes (24; 64; 84; 134, 144; 154) giving the person electric shocks.

7. The pulse sensor according to one of the preceding claims, characterized in that the radiation source comprises two different light sources, viz. an LED (21; 61; 81; 131, 141; 151) and an IRED (22; 62; 82; 132, 142; 152).

8. A pulse meter, comprising:
a pulse sensor according to claim 5 or claims 6 or 7, in as far as these are referred back to claim 5;
an evaluation circuit (25) supplied with the output signal of the photodetector (23; 63; 83; 133; 143; 153), for determining whether the pulse of the person is outside a permitted range (107), for controlling the radiation source (21, 22; 61, 62; 81, 82; 131, 132, 141, 142; 151, 152) and for activating the vibrator (43, 44, 45; 85) if the pulse of the person is outside the permitted range;
a housing (2, 3, 4) for accommodating the pulse sensor and the evaluation circuit, wherein the housing defines a battery case; and
a spring (43) fixed to one end of the battery case for pressing a battery (41; 47, 48) against a contact (46) on the opposite side of the battery case.

9. The pulse sensor according to claim 8, characterized in that the vibrator comprises:
a motor (44) driven by the evaluation circuit;
an eccentric (45) driven by the shaft of the motor (44); and
the contact (46) rotatably mounted on the eccentric (45) to press, if a battery (41) is inserted, the inserted battery (41) against the spring (43) so that, if a battery (41) is inserted and the eccentric (45) rotates, the spring (43) is periodically pressed together and the inserted battery (41) moves with respect to the battery case.

10. The pulse sensor according to claim 8 or 9, characterized in that the vibrator comprises:
a coil (49) generating a magnetic field when the coil (49) is flown through by a current, wherein the evaluation circuit (25) supplies an alternating voltage to the coil (49) if the pulse of the person is outside the permitted range (107); and
a magnet (50) on which the magnetic field of the coil exerts a force.

11. A pulse meter, comprising:
a pulse sensor according to one of claims 1 to 7;
an evaluation circuit (25) supplied with the output signal of the photodetector (23; 63; 83; 133; 143; 153), which controls the radiation source (21, 22; 61, 62; 81, 82; 131, 132, 141, 142; 151, 152) and is constructed to perform the following during operation:
determining the pulse of a person;
comparing the pulse with a total of four thresholds, viz. a lower warning threshold (105), a lower alarm threshold (106), an upper warning threshold (103) and an upper alarm threshold (104);
activating the stimulus generator (43, 44, 45; 85) in a warning mode if the pulse of the person is between the lower warning threshold (105) and the lower alarm threshold (106) or between the upper warning threshold (103) and the upper alarm threshold (104);
activating the stimulus generator (43, 44, 45; 85) in an alarm mode if the pulse of the person is above the upper alarm threshold (104) or below the lower alarm threshold (106).

12. An oximeter, comprising:
a pulse sensor according to claim 7;
an evaluation circuit (25) supplied with the output signal of the photodetector (23; 63; 83; 133; 143; 153), which controls the radiation source (21, 22; 61, 62; 81, 82; 131, 132, 141, 142; 151, 152) and is constructed to perform the following during operation:
determining the oxyhemoglobin content in the blood of a person;
comparing the oxyhemoglobin content with a threshold (94, 95);
activating the stimulus generator (43, 44, 45; 85) if the oxyhemoglobin content of the person is below the threshold (94, 95).

13. The oximeter according to claim 12, characterized in that the stimulus generator is a vibrator and that the vibrator comprises:
a coil (49) generating a magnetic field when the coil (49) is flown through by a current, wherein the evaluation circuit (25) supplies an alternating voltage to the coil (49) if the pulse of the person is outside the permitted range (107); and
a magnet (50) on which the magnetic field of the coil exerts a force.

14. A joystick (130), characterized by:
a radiation source (131, 132, 141, 142) for emitting photons;
a photodetector (133; 143) for detecting the photons, wherein, by holding the joystick by a person, the radiation source (131, 132, 141, 142) and the photodetector (133; 143) are fixed with respect to the fingers of the person; and
a connection (146) to a pulse meter, wherein the pulse meter controls the radiation source (131, 132, 141, 142) and reads out the photodetector (133; 143) via the connection (146).

15. The joystick according to claim 14, characterized by an stimulus generator (134, 135, 144, 145) for generating an stimulus attracting the attention of the person when the stimulus generator is activated by the pulse meter, wherein the stimulus generator (134, 135, 144, 145) is activated by the pulse meter when the pulse or the oxyhemoglobin content of the person are outside a permitted range (107).

16. A helmet (150), characterized by:
a radiation source (151, 152) for emitting photons;
a photodetector (153) for detecting the photons, wherein the radiation source (151, 152) and the photodetector (153) are fixed by the helmet with respect to the head of the person; and
a connection (165) to a pulse meter, wherein the pulse meter controls the radiation source (151, 152) and reads out the photodetector (153) via the connection (165).

17. The helmet (150) according to claim 16, characterized by an stimulus generator (154, 156) for generating an stimulus attracting the attention of the person when the stimulus generator is activated by the pulse meter, wherein the stimulus generator (154, 156) is activated by the pulse meter when the pulse of the person is outside a permitted range (107).
